(19) United States
(12) Patent Application Publication
(10) Pub. No.: US 2006/0286169 A1
(43) Pub. Date: Dec. 21, 2006

(54) Lipophilic compositions

(75) Inventors: Mathew Louis, Stephen Leigh, Basel (CH); Peter Van Hoogevest, Bubendorf (CH); Jacques Quinton, Waldighofen (FR); Steve Leigh, Amsterdam (NL)

Correspondence Address: BUCHANAN, INGERSOLL & ROONEY PC, POST OFFICE BOX 1404, ALEXANDRIA, VA, 22313-1404, US

(73) Assignee: Phares Pharmaceutical Research N.V., Curracao (AN) (NL)

(21) Appl. No.: 10/545,416

(22) PCT Filed: Feb. 13, 2004
(86) PCT No.: PCT/EP04/01355

§ 371 Date: Jun. 28, 2006

(30) Foreign Application Priority Data
Feb. 13, 2003 (EP) 03250900.2

(51) Int. Cl. A61K 9/14

(52) U.S. Cl. 424/486

(57) ABSTRACT

There is described dry compositions comprising lipophilic compounds associated with low viscosity grades of water insoluble polymer and optionally hydrophilic agent/s associated either as monomolecular or amorphous complexes. There is also described a method of preparing said lipophilic polymer complexes from a solution or homogeneous dispersion employing either water miscible or immiscible organic solvents. The lipophilic polymer complex is precipitated from the solution comprising a water miscible solvent by dilution with water, separating out the precipitated complex, washing, drying and conversion to oral and topical dosage forms. The lipophilic polymer complex may also be prepared by solvent removal involving spray drying or vacuum drying under elevated temperatures using either water miscible or water immiscible solvents. The compositions are characterised by improved dissolution and solubility of the associated compound in aqueous medium.

This invention relates to compositions and methods thereof for preparing pharmaceutical dosage forms comprising biologically active compounds with low water solubility complexed with water insoluble polymers to increase solubility in aqueous media.

### BACKGROUND OF THE INVENTION

Lipophilic compounds should be in the molecular state to allow optimum transport across membranes. Size reduction techniques employing intensive mechanical, fluid or ultra sound energy are extensively used to obtain fine powders, which have large surface areas exposed to dissolution medium. However, this alone may not be sufficient to increase solubility and reach bioavailability targets of at least 30%. Other methods used to increase solubility include derivitisation and formation of soluble salts, amorphous forms, molecular complexes, eutectics, solid solutions, self emulsifying compositions, etc.

Given the wide range of water insoluble compounds it is unlikely that one particular technology will be universally suitable to improve solubility. Hence there is a requirement for novel, industrially applicable methods for increasing dissolution of new and existing compounds without resorting to chemical modification or derivatisation.

### PRIOR ART

U.S. Pat. No. 4,992,278 describes compositions comprising drugs embedded in high viscosity grades of water insoluble polymers to modify the release of drugs with low solubility in the GI tract

WO 00/33817 describes hardened lipid compositions comprising either hydrophilic or lipophilic compounds with phospholipids and hydrocolloids in solid oral dosage forms.

J. Microencapsulation January-February 1996; 13(1), pp 89-98 describes micro-matrices comprising the lipophilic drug ketoprofen and solid blends of high viscosity 14 cP ethylcellulose and cellulose acetate trimellitate. It does not disclose compositions comprising a lipophilic drug and low viscosity lipophilic polymers without pH sensitive cellulose ester. Furthermore, the compositions do not require organic solvents to co-solubilise ethyl cellulose and the lipophilic compound in preparing the micro-matrix. J. Control Release 66(2000) 107-113, describes tramadol complexed with a resin. A suspension of the drug resin complex is coated by spray drying using ethylcellulose with viscosities between 10 and 100 cP. It does not disclose a drug complexed with ethyl cellulose.

U.S. Pat. No. 5,389,382 describes injectable liquid compositions comprising colloidal hydrosols of a lipophilic compound and ethyl cellulose suspended in water. A hydrosol is a sol that has water as its liquid phase and is outside the claims of the present invention which describe dry solid/particulate compositions for oral use. Furthermore in contrast to the present invention, the process described requires very rigorous conditions to form hydrosols suitable for injection and does not require separation /collection of the lipophilic complex and removal of water miscible solvent from the precipitate by washing and end drying.

Extant methods and compositions employed for improving dissolution and aqueous solubility result in complexes consisting of lipophilic compounds and essentially hydrophilic polymers. The references teach away from the invention and do not suggest a method of preparing oral compositions comprising lipophilic compounds complexed with water insoluble polymers and hydrophilic agents to improve solubility as well as compositions comprising lipophilic compounds complexed with low viscosity grades of ethyl cellulose and/or lipophilic resins for improving solubility.

### SUMMARY OF THE INVENTION

The invention is in the area of ‘lipophilic precipitates’ and ‘lipophilic polymer matrices’ or ‘lipophilic polymer complexes’ comprising compounds with low water solubility and essentially water insoluble polymers.

The invention relates to a method of preparing dry solid particles to increase the physical stability, dissolution rate and solubility of water insoluble compounds. Thus the invention describes methods and compositions thereof comprising lipophilic compounds homogeneously dispersed and associated in a dry /particulate polymeric matrix for use in oral or topical dosage forms.

Subject matter of the present invention is a method of preparing a pharmaceutical dosage form for oral or topical administration of a therapeutic agent with low water solubility, which method comprises,
a) Homogeneously dispersing the therapeutic agent and at least one lipophilic polymer in at least one water-miscible organic solvent, diluting the resulting solution with water, collecting the resulting precipitate and drying, or
b) Homogeneously dispersing the therapeutic agent in at least one water-immuscible or water miscible solvent and at least one lipophilic polymer, removing the solvent and collecting the dry precipitate obtained and, after performing either one of process steps a) or b) further processing the resulting powder into a pharmaceutical dosage form.

The residual water content of the particulate/solid lipophilic complex from either method is preferably below 10 wt %.

### DETAILED DESCRIPTION OF THE INVENTION

The following definitions apply in this specification:

‘Homogenously dispersed’ or ‘dissolved’ refers to colloidal or molecular distribution of the component/s in a medium, matrix or solvent.

‘Pharmaceutical dosage form’ for oral or topical administration include hard gelatine powder capsules, soft gelatine capsules and their likes, tablets, powders for (DPI) dry powder inhalers, paste like creams, ointments and gels.

“Compounds” are biologically active substances that have a physiological and/or pharmacological effect. They are also referred to as drugs or agents and include nutriceuticals, feed components, cosmetic and diagnostic substances.

“A compound of low water solubility” includes any compound that requires more than 10 parts of water at pH 7 to dissolve 1 part of the compound or excipient. It spans the definitions between sparingly soluble (from 10 to 30) to very slightly soluble (from 1000 to 10′000) and practically insoluble or insoluble (10 000 and over) as defined in USP 24.

“Low viscosity” defines the viscosity of a 5% solution of a polymer (previously dried 30 min at 100° C.) in a solution of toluene/ethanol 80/20 w/w wherein the viscosity is equal or lower then 11 cP.

“Molecular” and “amorphous” define the distribution states of poorly water soluble compounds in the polymer complex whereby at least 50% of the compound transfers to a dissolution medium described in USP or an appropriate dissolution medium at 37° C. within 18 hours.

‘Monomolecular’ refers to a uniform distribution of molecules throughout a medium or matrix.

“Polymer” includes high molecular wt natural and synthetic compounds and excipients with repetitive units. The definition also includes resins and rosins.

“Water insoluble”, “lipophilic” and “hydrophobic” polymers are used synonymously in this specification. The terms include polymers that are practically insoluble in water; partially soluble in volatile water miscible or hydrophilic solvents such as ethanol; freely soluble in volatile lipophilic solvents such as methylene chloride; non-volatile hydrophilic solvents particularly N-methyl pyrollidone (NMP); mixtures of hydrophilic solvents and water.

“Water-miscible” or “hydrophilic” solvent refers to an organic liquid that can be diluted with at least an equal part of water without separation.

“Water-immiscible” or “lipophilic” solvent refers to an organic liquid that can not be diluted with at least an equal part of water without separation.

“Lipophilic polymer complex”, “lipophilic matrix” and “precipitates” are particulate compositions comprising compounds with low water solubility in molecular association either as monomolecular species, or as amorphous particles complexed in a polymeric matrix. The particle size range of the complex is between 5 μm to 500 μm. The compound may be in monomolecular distribution in the lipophilic polymer or it may be associated as amorphous particles. The compound dissociates easily from the complex comprising low viscosity water insoluble polymer which may contain a minor amount of hydrophilic agent.

As used herein, the terms ‘a’, ‘an’ and ‘any’ are intended to include both the singular and plural terms.

Precipitates prepared using lipophilic compounds and preferably an excess of lipophilic polymers homogeneously dispersed/dissolved in suitable solvents form molecular complexes when diluted with water or if the solvent is removed. Typically, depending on the physico-chemical properties of the compound and/or ratio of drug substance to polymer, the compound in the precipitated lipophilic matrix is complexed either monomolecularly or forms particulate amorphous associates embedded in the polymer matrix. Generally the particle size range of the associates is between 5 μm to 500 μm. In some cases, a small but consistent fraction of the drug content, up to 10% by weight may be crystaline, whilst about 90% are in molecular complex. Be it monomolecular association and/or amorphous formation, the compound is likely to show a faster dissolution rate due to weak hydrophobic bonding between lipophilic components. For instance, 98 wt % of a highly insoluble HIV compound complexed with a low viscosity grade of a lipophilic polymer such as ethyl cellulose is released from a lipophilic matrix within 6 hours.

Furthermore, the lipophilic polymer matrix helps to stabilise amorphous particles against excess moisture and prevents or delays crystallisation. Preferred lipophilic polymers for this purpose are low viscosity grades of polymers which unexpectedly allow the compound to disassociate more easily either from a molecular state or from amorphous particles, into aqueous media. This contrasts with compositions for improving solubility of lipophilic compounds using hydrophilic colloids. The lipophilic compound is more likely to separate out as insoluble crystals from a hydrophilic matrix.

It should be clearly understood that the invention relates to a method of preparation and to dry solid or particulate compositions thereof comprising lipophilic compounds that have improved solubility and dissolution properties for use in solid oral dosage or semi-solid topical forms. Depending on the hydrophobicity of the lipophilic compound, the compositions may optionally comprise smaller amounts of hydrophilic agents or polymers relative to the low viscosity lipophilic polymers including but not limited to surfactants, sugars and hydrophilic and osmotic components that dissolve easily in water. Since the compound may easily dissociate from the lipophilic matrix because of hydrophobic bonds, transfer to lipophilic regions lining mucosal absorption surfaces may be more efficient. The water content of the lipophilic polymer complex is preferably less than 10% by weight, preferably less than 5 wt %. The compositions are suitable in oral dosage forms for increasing the dissolution rate of lipophilic drugs by complex formation either in the molecular or amorphous states. By selecting the most appropriate grade, viscosity and polymer to drug ratio, the dissolution period of the lipophilic compound from the polymer complex may be extended for up to 12-18 hours, synchronizing with the desired absorption window of the drug, thereby maximizing oral absorption. The compositions may also be suitable for topical use in dermatological as well as mucosal applications including dry powder inhalation and nasal delivery.

In one aspect, the invention describes a method of preparing a composition comprising a lipophilic polymer complex which comprises in Process variant A;
i) homogeneously dispersing a therapeutic compound with low water solubility, a lipophilic polymer and optionally a hydrophilic agent in at least one water miscible organic solvent;
ii) diluting the resulting solution/molecular dispersion with water;
iii) collecting the resulting co-precipitate and drying;
iv) further processing the resulting powder into a pharmaceutical dosage form.

According to a preferred embodiment the therapeutic compound with low water solubility, the lipophilic polymer and optionally the hydrophilic agent are dissolved in at least one water miscible organic solvent, preferably N-methylpyrrolidone, ethanol, methanol, isopropanol or mixtures thereof. Typical of the method is that water miscible solvent with high (>100° C.) as well as low (<100° C.) boiling points at atmospheric pressure may be used.

The compound is firstly dissolved in the water miscible organic solvent together with at least one lipophilic polymer such as ethylcellulose and/or Dammar gum, optionally minor amounts of at least one water soluble agent, optionally using flash heating procedures for maximum solution. Secondly, the solution is diluted with water. The resulting precipitate may be washed with water to remove the solvent, dried and powdered if necessary. The particle size of the dry powder or particulate composition is below 1000 μM, preferably below 500 μm more preferably between 5 μm to 100 μm. However, they may be milled to obtain particles which are smaller than 5 μm for more rapid dissolution in oral dosage forms or for topical applications, particularly inhalation. The powder may also be agglomerated into granules suitable for capsule or sachet filing. Using suitable excipients and disintegrants the granules may be further processed into tablets. Preferably, hydrophilic solvents such as NMP and/or ethanol, isopropanol, and methanol with much less environmental toxicity than e.g. methylene chloride, are particularly suitable for preparing co-precipitates by addition of water and drying the polymer complex with or without heat and vacuum assistance and further particle size reduction.

Suitable water miscible organic solvents that may be removed by washing the precipitate are N-methyl-pyrrolidone (NMP), ethyl lactate and glycofurol and combinations thereof with water miscible solvents such as ethanol, methanol, acetone, etc.

The alternative Process variant B according to the invention comprises,
i) homogeneously dispersing compound with low water solubility, lipophilic polymer and optionally hydrophilic agent in at least one water miscible or immiscible solvent,
ii) removing the solvent,
iii) collecting the resulting dry precipitate,
iv) further processing the resulting powder into a pharmaceutical dosage form.

Accordingly, it may be more expedient to prepare lipophilic powder complexes directly by dispersing the components in water miscible or water immiscible organic solvent/s followed by removal of solvent/s by e.g. spray drying, spray granulation, or a similar process thereby yielding a solid lipophilic complex with similar properties to those obtained as if a water miscible solven had been employed in Process A. Suitable water miscible solvents that are also suitable for solvent removal processes such as spray drying, spray granulation, or vacuum drying are e.g. acetone, methanol, ethenol, isopropanol, n-propanol, NMP and mixtures thereof. Suitable water immiscible organic solvents that may be more amenable to solvent removal processes such as spray drying given strict solvent recovery installations are dichloromethane and dimethoxymethane, diethoxymethane and dioxacyclopentane.

The present invention also relates to a pharmaceutical composition comprising a therapeutic agent of low water solubility, at least one lipophilic polymer, and, optionally conventional pharmaceutical additives, which are water-soluble or have wetting properties, as obtained by the methods a) or b) as described above.

According to a preferred embodiment the present invention relates to a pharmaceutical composition comprising a therapeutic agent of low water solubility, at least one lipophilic polymer selected from the group consisting of low viscosity ethyl cellulose or a lipophilic resin of natural origin or a mixture of both, and, optionally conventional pharmaceutical additives.

Lipophilic compounds which may benefit from the invention are sparingly soluble therapeutic agents suitable for oral and topical administration. They are, e.g. immunosuppressants having a macrolide structure, typically cydosporin A, cyclosporin G, rapamycin, tacrolimus, deoxyspergualin, mycophenolate-mofetil, gusperimus; non-steroidal antiphlogistic agents and salts thereof, typically acetylsalicylic acid, ibuprofen or S(+)-ibuprofen, indomethacin, diclofenac (Na and K-salt), piroxicam, meloxicam, tenoxicam, naproxen, ketoprofen, flurbiprofen, fenoprofen, felbinac, sulindac, etodolac, oxyphenbutazone, phenylbutazone, nabumetone COX-2 inhibitors like celcoxib and steroidal antiflogistics like prednisone, cortisone; dihydropyridine derivatives having cardiovascular activity, e.g. nifedipine, nitrendipine, nimodipine, nisoldipine, isradipine, felodipine, amlodipine, nilvadipine, lacidipine, benidipine, masnipine, furnidipine, niguldipine; angiotensin II receptor antagonists like candesartan, lipophilic anticoagulants; thrombolytics; immunodepressants and stimulants, typically a-liponic acid; CNS acting agents, e.g. reserpine, ergot alkaloids, typically bromocriptine, dihydroergotarine, dihydroergocristine; carbamazepine, imipramine, benzodiazepines, nicotine, caffeine; antitumour agents, e.g. vincopectin, vincristine, vinblastin, chlorambucil, etoposide, teniposide, idoxifen, timustin, teloxantron, tirapazamine, carzelesin, dexniguldipine, intoplicin, idarubicin, miltefosin, trofosfamide, teloxantrone, melphalan, lomustine, 4,5-bis(4′fluoroanilino)phthalimide; 4,5-dianilinophthalimide; immunomodulators like tacrolimus, typically thymoctonan, prezatid copper acetate; H2-receptor antagonists, typically famotidine, cimetidine, ranitidine, roxatidine, nizatidine, omeprazole, proteinkinase inhibitors; or HIV-1 or HIV-2 protease inhibitors or leucotriene antagonists, lipophilic narcotics/aneasthetics like propofol and hormones like estrogen, testosterone and their esters.

Instead of the free acid or base, the therapeutic agents may be converted to a salt, typically as hydrobromide, hydrochloride, mesylate, acetate, succinate, lactate, tartrate, fumarate, sulfate, maleate, and the like, especially if the counter ions form ion-pair which is solvent soluble.

To increase the stability of the active compound against oxidation, it is advantageous to add lipophilic stabilizers such as alpha-tocopherol, t-butylated hydroxytoluene, t-butylated hydroxyanisole or ethoxyquin. Preferably they are dissolved together with the compound and other lipophilic excipient in the organic phase.

Typical examples of lipophilic polymers or excipients used in this invention are water insoluble polymers such as ethylcellulose (Dow Chemical, USA; Hercules, FRG) and Dammar gum (CNI; France). Preferred are ethylcellulose grades with not less than 44% and not more than 51% by weight of ethoxy groups. More preferred are cellulose grades meeting the requirements of the National Formulary of 48.0-49.5% ethoxy group content (N-grade) (Hercules, Product data brochure on AQUALON® Ethylcellulose). In some cases, however, cellulose grades with less than 44% or more than 51% by weight may be used as well. Depending on the molecular weights, the grade of cellulose may have viscosities up to 11 cps. Most preferable are ethylcellulose grades with low viscosity, such as N7 or N4 or lower such as N3, obtainable from Dow Chemical or Hercules Inc. The values are obtained from a 5% w/w solution comprising 80 parts toluene and 20 parts ethanol.

Dammar gum is a resin characterised by low viscosity and is a preferred alternative to low viscosity ethylcellulose. Dammar gum can also be used in combination with low viscosity ethylcellulose. The following resins can be used either as the lipophilic excipient on its own or in combination with low viscosity N grade ethylcellulose: A) Natural resins: Batu run resin, Congo run resin, Elemi resin, Kauri resin, Manila gum, Mastic gum, Rosin wood resin, Sandarc resin, shellac resin, white shellac, Vinsol® resin; B) Rosin and terpene base resins: Abalyn, Abitol E, Cellolyn 21 102M, Ester gum, Hercolyn D, Lewisol 28, Pentalyn A, H, 830. 856, Pentrex 28, Poly paleresin, Stabelite 3, 10 ester Vinsol ester gum, Zinar, Zirex and Zitro, Uni-Rez 7200;

The ratio of lipophilic compound to the lipophilic polymer is between 1:200 to 10:1. Preferably it is 1:1 to 1:50. more preferably it is 1:1 to 1:10.

Suitable organic solvents may be water immiscible or preferably water miscible solvents; Examples of water immiscible organic solvents are, methylene chloride, dimethoxymethane etc and mixtures thereof. Examples of preferred water miscible solvents are, e.g. NMP, isopropanol, ethanol, 96% ethanol, methanol ethyl lactate, polyethylene glycol 300, polyethylene glycol 400, 1,2 propanediol, 1,3 butanediol, succinic acid diethyl ester, triethyl citrate, dibutyl sebacate, dimethyl acetamide, DMSO, glycerineformal, glycofurol(tetraglycol), isopropanol, lactic acid butyl ester, propylene carbonate, propylene glycol diacetate, tetrahydrofurfuryl alcohol, diethylene glycol mono ethyl ether and mixtures thereof.

The invention allows the co-precipitates preferably to include smaller amounts of hydrophilic excipients and ingredients. These confer or have wetting properties and/or are highly water soluble. Examples are PEG (polyethylengylcol) with MW 4000-6000, polyvinylpyrrolidone, polyvinylalcohol, crosspovidone, polyvinylpyrrolidone-polyvinylacetate copolymer, cellulose derivatives, like hydroxypropylmethylcellulose (HMPC), hydroxypropylcellulose (HPC), hydroxypropylmethylcellulose phthalate (HPMCP), polyacrylates and polymethacrylates, natural hydrocolloids, gelatine, urea, sugars, polylols, chitosan, organic acids (succinic acid, citric acid) and non ionic, anionic, cationic or amphoteric surfactants such as phospholipids.

The weight ratio of low viscosity grades of lipophilic polymer to hydrophilic polymer or agent/s in the composition may be between 2:1 to 10:0.1. Preferably from 5:1 to 10:1.

The compositions according to the invention are eminently suitable as pharmaceutical dosage forms and in food (including nutriceuticals) and feed formulations. The powder complex may be used as such or incorporated directly into food and feed applications.

For oral dosage forms, the dry powder complex or granules may be blended with suitable bulking agents and flow aids to the required fill weight for hard gelatine capsules or the like. Optionally, the powder complex may be formulated with disintegrants and compression aids for compaction into tablets.

For pulmonary or nasal delivery, the dry powder complex may be micronised to below 10 μm, preferably between 2 μm to 7 μm weight mean particle diameter for delivery of lipophilic compounds in (DP) dry powder inhalers.

The micronised powder complex may be suspended in creams and ointments and other non aqueous systems for dermatological applications. Suitable non aqueous vehicles are eg. polyols, PEGS or fatty acid glycerides, esters and ethers, etc.

There is described dry compositions comprising lipophilic compounds associated with low viscosity grades of water insoluble polymer and optionally hydrophilic agent/s associated either as monomolecular or amorphous complexes. There is also described a method of preparing said lipophilic polymer complexes from a solution or homogeneous dispersion employing either water miscible or immiscible organic solvents. The lipophilic polymer complex is precipitated from the solution comprising a water miscible solvent by dilution with water, separating out the precipitated complex, washing, drying and conversion to oral and topical dosage forms. The lipophilic polymer complex may also be prepared by solvent removal involving spray drying or vacuum drying under elevated temperatures using either water miscible or water immiscible solvents. The compositions are characterised by improved dissolution and solubility of the associated compound in aqueous medium.

The following examples illustrate the invention.

### EXAMPLE 1

167 mg of an anti HIV compound (solubility in water <0.010 μg/ml), 167 mg of HPMC and 1667 mg Ethylcellulose N4 (Dow Chemical) are dissolved in 10 ml ethanol/NMP (50/50 v/v). The dear solution is added under siring to a ten fold excess of water to prepare particulate precipitates that are below 500 μm. The resulting precipitate is collected on a filter and washed twice with 20 g water to remove the solvents. The wet mass is dried for 8 h at 30° C. in a vacuum oven. The resulting free flowing fine powder is used to fill hard gelatine capsules, suitable for oral administration.

### EXAMPLE 2

Similar to Example 1, in place of 167 mg HPMC, 167 mg alpha tocopherol is used.

### EXAMPLE 3

Similar to Example 2, in place of Ethylcellulose N4, Ethylcellullose N3 (Hercules) is used.

### EXAMPLE 4

Similar to Example 3, in place of Ethylcellulose N4, Ethylcellullose N7 (Dow) is used.

### EXAMPLE 5

167 mg of an anti HIV compound used in Example 1, 167 mg of HPMC and 1667 mg Dammar gum (NCI) are dissolved in 10 ml methylene chloride. The dear solution is added to a fluidised bed dryer or spray granulator or spray dryer and the solvent is removed at 30° C. during 4 hours. The resulting free flowing fine powder is used to fill hard gelatine capsules, suitable for oral administration.

### EXAMPLE 6

100 mg of nifedipine, 1000 mg Ethylcellulose N3 (Hercules) are dissolved in 10 ml NMP. The clear solution is added under stirring to a ten fold excess of water. The resulting precipitate is collected on a filter and washed twice with 20 g water to remove the solvent. The wet mass is dried for 8 h at 30° C. in a vacuum oven. The resulting free flowing fine powder is used to fill hard gelatine capsules, suitable for oral administration.

### EXAMPLE 7

Analogue to Example 6, instead of nifedipine 100 mg candesartan is used.

### EXAMPLE 8

Analogously to Example 6, instead of Nifedipine 100 mg celcoxib is used.

1-10. (canceled)

11. A composition for oral or topical administration of a therapeutic compound with low water solubility as obtained by a solvent removal process which comprises,
a) homogeneously dispersing,
i)) the therapeutic agent and,
ii) at least one lipophilic polymer in
iii) at least one water miscible or water immiscible solvent, and,
b) removing the solvent either by diluting the resulting solution with water, collecting the resulting precipitate and drying, or removing the solvent by spray drying, spray granulation, or a similar process yielding a solid lipophilic complex, and,
c) further processing the resulting powder into a pharmaceutical dosage form.

12. A composition according to claim 11 wherein the lipophilic polymer is selected from the group consisting of low viscosity ethyl cellulose with viscosities up to 11 cP, Dammar gum, lipophilic resins of natural origin, Rosin and terpene base resins, and mixtures of said lipiphilic polymers with said low viscosity ethyl cellulose.

13. A composition according to claim 11 wherein the water-miscible organic solvent is selected from the group consisting of NMP, isopropanol, ethanol, 96% ethanol, methanol, ethyl lactate, polyethylene glycol 300, polyethylene glycol 400, 1,2 propanediol, 1,3 butandiol, succinic acid diethyl ester, triethyl citrate, dibutyl sebacate, dimethyl acetamide, DMSO, glycerineformal, glycofurol (tetraglycol), isopropanol, lactic acid butyl ester, propylene carbonate, propylene glycol diacetate, tetrahydrofurfuryl alcohol, diethylene glycol mono ethyl ether and mixtures thereof.

14. A composition according to claim 11 wherein the water-immiscible organic solvent is selected from the group consisting of dichloromethane and dimethoxymethane, diethoxymethane and dioxacyclopentane.

15. The composition of claim 11 which is a pharmaceutical dosage form.

16. The composition of claim 11 wherein the particle size range of the lipophilic complex is between 5 μm to 500 μm.

17. The composition of claim 11 wherein said composition comprises monomolecular associates of said therapeutic agent with low water solubility.

18. The compositon of claim 11 wherein said composition comprises particulate amorphous associates of said therapeutic agent with low water solubility between 5 μm to 500 μm.

19. The composition of claim 11 wherein said composition comprises monomolecular and amorphous associates of said therapeutic agent with low water solubility.

20. The composition of claim 11 comprising up to 90 wt % of the total amount present of said therapeutic agent with low water solubility in monomolecular and amorphous association.

21. A method for preparing a composition for oral or topical administration of a therapeutic compound with low water solubility, to increase the physical stability, dissolution rate and solubility of said therapeutic compound, said method comprising
a) homogeneously dispersing,
i)) the therapeutic agent and,
ii) at least one lipophilic polymer in
iii) at least one water miscible or water immiscible solvent, and,
b) removing the solvent either by diluting the resulting solution with water, collecting the resulting precipitate and drying, or removing the solvent by spray drying, spray granulation, or a similar process yielding a solid lipophilic complex, and,
c) further processing the resulting powder into a pharmaceutical dosage form.

22. The method of claim 21 further including the addition of pharmaceutical additives which are water-soluble or have wetting properties, said pharmaceutical additives being selected from the group consisting of PEG (polyethylengylcol) with MW 4000-6000, polyvinylpyrrolidone, polyvinylalcohol, crosspovidone, polyvinylpyrrolidone-polyvinylacetate copolymer, cellulose derivatives, like hydroxypropylmethylcellulose (HMPC), hydroxypropylcellulose (HPC), hydroxypropylmethylcellulose phthalate (HPMCP), polyacrylates and polymethacrylates, natural hydrocolloids, gelatine, urea, sugars, polylols, chitosan, organic acids (succinic acid, citric acid) and non ionic, anionic, cationic or amphoteric surfactants such as phospholipids.

23. A composition according to claim 12 wherein the water-miscible organic solvent is selected from the group consisting of NMP, isopropanol, ethanol, 96% ethanol, methanol, ethyl lactate, polyethylene glycol 300, polyethylene glycol 400, 1,2 propanediol, 1,3 butandiol, succinic acid diethyl ester, triethyl citrate, dibutyl sebacate, dimethyl acetamide, DMSO, glycerineformal, glycofurol(tetraglycol), isopropanol, lactic acid butyl ester, propylene carbonate, propylene glycol diacetate, tetrahydrofurfuryl alcohol, diethylene glycol mono ethyl ether and mixtures thereof.

24. A composition according to claim 12 wherein the water-immiscible organic solvent is selected from the group consisting of dichloromethane and dimethoxymethane, diethoxymethane and dioxacyclopentane.
